(19) United States
(12) Patent Application Publication
(10) Pub. No.: US 2013/0204315 A1
(43) Pub. Date: Aug. 8, 2013

(54) SYSTEMS FOR AND METHODS OF TRANSCRANIAL DIRECT CURRENT ELECTRICAL STIMULATION

(71) Applicants: AMORN WONGSARNPIGOON, Mebane, NC (US); Joseph W. Boggs, II, Carrboro, NC (US); Stuart F. Rubin, Orange Village, OH (US); Jonathan L. Sakai, Fairview Park, OH (US)

(72) Inventors: AMORN WONGSARNPIGOON, Mebane, NC (US); Joseph W. Boggs, II, Carrboro, NC (US); Stuart F. Rubin, Orange Village, OH (US); Jonathan L. Sakai, Fairview Park, OH (US)

(73) Assignee: NDI MEDICAL, LLC, CLEVELAND, OH (US)

(21) Appl. No.: 13/567,716

(22) Filed: Aug. 6, 2012

Related U.S. Application Data
(60) Provisional application No. 61/515,492, filed on Aug. 5, 2011.

(51) Int. Cl. A61N 1/36

(52) U.S. Cl. CPC A61N 1/36025; A61N 1/36032; A61N 1/36021
USPC 607/45; 607/55; 607/46

(57) ABSTRACT

A system according to the present invention provides a portable, non-invasive device adapted to deliver electrical stimulation to a brain, such as to treat tinnitus. Such system is preferably a head-worn system configured to provide transcranial direct current electrical stimulation (tDCS) to a patient, where a therapy based at least partially thereon may be self-administered by the patient. tDCS is a non-invasive method of brain stimulation to treat tinnitus, or other neurological indications, that may provide significant relief. Methods according to the present invention include preferably brief sessions of anodal tDCS to assist in determining adequate electrode location and stimulus intensity by producing transient decreases in tinnitus intensity. Methods may also or alternatively include a number of sessions of cathodal tDCS at a confirmed electrode location and stimulus intensity to provide sustained tinnitus relief. Methods may also or alternatively include a number of maintenance sessions to prolong the sustained relief.

### RELATED APPLICATIONS

This application claims the benefit of co-pending U.S. Provisional Patent Application Ser. No. 61/515,492, filed 5 Aug. 2011, and entitled “Systems for and Methods of Transcranial Direct Current Electrical Stimulation.”

### BACKGROUND OF THE INVENTION

Embodiments of the present invention relate to systems and methods of electrical stimulation applied to an animal, and more specifically a portable, non-invasive system to electrically stimulate the brain, to provide treatment for indications such as tinnitus, epilepsy, addiction, depression, stroke, anorexia, pain, and/or the improvement of attention and/or motor learning.

In particular, the present invention can be used to treat tinnitus. Tinnitus is a disorder where sounds (e.g. ringing, hissing, clicking) are perceived without an external source. Approximately 3-9 million people (1-3% of the population) in the U.S. suffer from severe and persistent tinnitus, greatly reducing quality of life (e.g. sleeping disorders, anxiety, depression). These symptoms often force tinnitus sufferers to make significant adjustments, including avoiding everyday activities, hobbies, and important life events. In extreme cases, tinnitus has led to suicide.

There is presently no cure for tinnitus and most patients do not benefit from present treatments. Many therapies aim to help patients cope with tinnitus but are often unsuccessful and do not reduce the perception of sound. Drugs provide limited and/or transient relief of symptoms, are not FDA approved for the treatment of tinnitus, and typically produce side effects. Other therapies are invasive (e.g., chronic neural stimulation), lack clinically meaningful data (e.g., Neuromonics), and/or require frequent visits to treatment centers (e.g., repetitive transcranial magnetic stimulation), and collectively have shown limited efficacy.

Present methods of cortical stimulation to treat tinnitus or other indications suggesting such stimulation are inconvenient, invasive, produce sustained relief in only a minority of patients, and/or lack simple and accurate methods of determining correct electrode positioning. Repetitive transcranial magnetic stimulation is non-invasive and reduces tinnitus by modulating cortical excitability, but treatment is prohibitively inconvenient and it has been reported that only a minority of patients have sustained relief. Studies of repetitive transcranial magnetic stimulation (rTMS) to treat tinnitus have demonstrated the potential for non-invasive cortical stimulation to provide sustained relief of tinnitus. Repeated daily sessions (5-10 days) of rTMS result in partial to total relief of tinnitus symptoms for ≧2 days in ˜20-65% of patients, but relief is sustained (3-12 months) for only a minority of patients (21-42%). Due to the size and cost of rTMS devices, rTMS can only be administered at a treatment center. To prolong tinnitus relief, patients must return to the treatment center for maintenance sessions, which can be inconvenient, expensive, and time-consuming (particularly for patients living in rural areas far from treatment centers), and patient compliance decreases with travel distance for many outpatient therapies. Although portable magnetic stimulators have been developed, these devices are not FDA approved and do not deliver the repeated pulses that have been demonstrated to reduce tinnitus. Although uncommon, seizure induction is a risk with rTMS.

Furthermore, methods to determine the correct position of the rTMS coils are expensive, time-consuming, and/or inaccurate. Studies of rTMS to treat tinnitus have employed functional neuroimaging (functional magnetic resonance imaging [fMRI], positron emission tomography [PET], single-photon emission computed tomography [SPECT]), and coils were positioned over hyperactive regions of the cortex of tinnitus patients to disrupt or reduce the hyperactivity, and hence, reduce tinnitus. Although functional neuroimaging can identify cortical locations accurately, its clinical use is limited because it is expensive, time-consuming, uses radioactive agents (for PET and SPECT), and requires multiple personnel (e.g., radiologists, technicians). Another method to position rTMS coils has relied only on an anatomical landmark-based system. Although this method can be performed more quickly and cheaply than functional neuroimaging, the accuracy of this method is less reliable; estimated cortical locations can be off target by up to 20 millimeters (mm) and measurement errors can lead to errors of 7 mm, reducing the probability of efficacy. The overall rate of success for rTMS treatment of tinnitus is less than 40% for either landmark-based or functional imaging-based electrode placement.

Because rTMS produces sustained relief in only a minority of patients, is not readily accessible or inaccurate or expensive and time-consuming methods of coil positioning, the adoption of rTMS has been limited.

Auditory cortex stimulation (ACS) is an investigational chronic treatment for tinnitus, where an electrode is implanted beneath the skull, and the electrode is connected to a battery-powered implanted pulse generator (IPG). Auditory cortex stimulation targeting hyperactive cortical regions can reduce tinnitus but requires invasive, expensive surgery and risks infection and other complications. Across all studies of ACS to treat tinnitus, 77% of patients using ACS experienced 25-100% reduction in tinnitus intensity. Also, because the device is implanted, patients are not required to travel to treatment centers for stimulation. However, surgeries for the implantation of the electrode and IPG, as well as replacement of the IPG when the battery is depleted, are expensive, invasive, and carry risks of complications (e.g., infections, hematoma, cerebral hemorrhage). Also, in one study 3/43 patients (7%) experienced seizures. Thus, despite promising initial results, the risks and costs of ACS have limited its potential as a treatment for tinnitus.

Transcranial direct current stimulation (tDCS) is an investigational non-invasive therapy that may be used for the treatment of tinnitus, epilepsy, addiction, depression, pain, and/or other indications. Existing methods and systems of tDCS involve the application of relatively weak constant current to the scalp via liquid-soaked sponge electrodes connected to an external stimulator.

Thus, unlike ACS, tDCS does not require invasive surgery and avoids the associated risks and costs. Unlike rTMS, tDCS can be delivered using a portable device at home, and treatment can be delivered without the substantial time and cost associated with traveling to treatment centers. Further, unlike both rTMS and ACS, there are no known reports of seizures caused by tDCS.

Despite these advantages of tDCS over ACS and rTMS, present methods of tDCS for the treatment of tinnitus have produced only moderate benefit. Transient reduction of tinnitus intensity, ranging from slight to complete, was produced in 30-47% of patients. Only one known study has reported long-term effects of tDCS on tinnitus, and results were inconsistent: 35% of subjects experienced improvements lasting for hours to ≧15 days, while 20% of subjects experienced negative effects. The present methods of tDCS for tinnitus could be modified to improve the outcome. Specifically, the amplitude, number and duration of sessions, and interval between sessions could be altered to increase the size and duration of the tinnitus relief, which would render tDCS more clinically viable.

tDCS may benefit from an improved method for determining correct electrode position for delivering stimulation. As with studies of rTMS, present methods of tDCS for tinnitus rely solely on a landmark-based system for electrode placement, which alone is inaccurate and may lead to low response rates. A quick, simple, inexpensive, and accurate method to determine correct electrode position that can be performed by a single clinician or a patient may improve the consistency of success of tDCS and the probability of acceptance of tDCS by patients and clinicians.

Thus, while tDCS is a non-invasive method of brain stimulation to treat tinnitus and other indications that has minimal side effects, existing methods to determine correct electrode position are either inaccurate, expensive, and/or time-consuming. Although tDCS can produce transient relief of tinnitus, the ability to generate reliable sustained relief of tinnitus has not yet been demonstrated by prior devices and methods. A simple but accurate method of determining correct electrode position, as well as demonstrated sustained relief, are desirable to make this promising portable therapy clinically viable.

### SUMMARY OF THE INVENTION

Methods according to the present invention may provide sustained relief of, for example, tinnitus using tDCS of, for example, the left temporoparietal area (LTA). The methods employed may produce a transient decrease in symptom intensity, or alternatively, could be used to generate an alternative functional or non-functional response, to identify correct electrode position to be targeted for additional treatment sessions of cathodal tDCS. Furthermore, a number, e.g. 5-30 of daily sessions, or weekly or monthly sessions, of prolonged cathodal tDCS may result in sustained clinically significant relief. Generally, as used herein, cathodal tDCS is defined as a primary electrode serving as a cathode, establishing a net negative charge at a stimulation location, and disposed at a lower electrical potential than a return electrode. Further, as used herein, anodal tDCS is defined as a primary electrode serving as an anode, establishing a net positive charge at a stimulation location, and disposed at a higher electrical potential than a return electrode.

Methods according to the present invention include sessions of anodal tDCS delivered over an area of the brain, such as the LTA, which has been shown to be hyperactive in 85-90% of tinnitus patients. Such stimulation may transiently decrease tinnitus intensity. The location of the LTA, or other region of the brain, may be approximated using a landmark-based system, and electrode location may be confirmed using anodal tDCS. Anodal tDCS may be delivered around the desired location for a predetermined time, such as about 3 minutes. If a patient experiences a decrease in the indication or experiences another response, such as a functional or non-functional response, or specifically, a Numerical Rating Scale (NRS) for tinnitus intensity decrease by a predetermined percentage, such as 50%, immediately following stimulation, electrode placement may be noted, logged, and/or secured.

Further, daily sessions, either short (e.g. 1-5 minutes) or prolonged (i.e. >5 mins), of cathodal tDCS of the LTA, or other effective area, may provide sustained relief. Patient selection and/or screening for such therapy may be tested by measuring, for example, tinnitus distress before and after daily sessions of tDCS on a predetermined number, such as 2 to 30, of consecutive days, or after a predetermined number of weekly or monthly sessions, and determining if a) the patient experiences a minimum (e.g. ≧10) point reduction on a standard Tinnitus Questionnaire lasting a minimum number (e.g. ≧7) days following the final session, or b) this reduction was significantly greater than placebo. The improvement in symptoms may last much longer than the minimum number of days (e.g. longer than the duration of the treatment). It is possible that a short-duration treatment may produce a long-duration effect (e.g. approximately 30 days of treatment may produce approximately 90 days or more or improvement).

Systems according to the present invention provide novel technologies that allow tDCS to be delivered quickly and consistently to any location on the scalp, preferably without the need to measure and/or confirm electrode locations before each session, thus improving the use of tDCS as an at-home treatment, or otherwise without clinician intervention (see FIG. 1).

### BRIEF DESCRIPTION OF THE DRAWINGS

FIG. 1 is a top view of the 10-20 EEG system, showing the coordinates that may be referenced for positioning electrodes and receiving stimulation according to the present invention.

FIG. 2A is an exploded perspective view of a first embodiment of an electrode according to the present invention.

FIG. 2B is a perspective view of the first embodiment of an electrode used according to the present invention shown in FIG. 2A.

FIG. 3A is an exploded perspective view of a second embodiment of an electrode according to the present invention.

FIG. 3B is a perspective view of the second embodiment of an electrode used according to the present invention shown in FIG. 3A.

FIG. 4 is a front elevation view of a person having an embodiment of a tDCS system according to the present invention positioned on his head.

FIG. 5 is a top plan view of the positioning of FIG. 4, further schematically imposing the 10-20 EEG system from FIG. 1, showing an electrode according to the present invention positioned near the LTA.

FIG. 6A is a perspective view of an embodiment of a tDCS system according to the present invention, shown in a first position.

FIG. 6B is a perspective view of the embodiment of the tDCS system shown in FIG. 6A, shown in a second position.

FIG. 6C is a perspective view of the embodiment of the tDCS system shown in FIG. 6A, shown in a third position.

FIG. 7 is a perspective view of an electrode attachment mechanism according to the present invention.

FIG. 8 is a bottom perspective view of a third embodiment of an electrode according to the present invention.

FIG. 9 is a front elevation view of a person having a second embodiment of a tDCS system according to the present invention positioned on his head.

FIG. 10 is a front elevation view of a person having a third embodiment of a tDCS system according to the present invention positioned on his head.

FIG. 11 is a front view of a control panel according to the present invention.

FIG. 12 is a rear view of a person utilizing the tDCS system of FIG. 10.

FIG. 13 is a perspective view of an aspect of the tDCS system according to the present invention as it may be positioned during use.

### DESCRIPTION OF THE PREFERRED EMBODIMENT

Although the disclosure hereof is detailed and exact to enable those skilled in the art to practice the invention, the physical embodiments herein disclosed merely exemplify the invention which may be embodied in other specific structures. While the preferred embodiment has been described, the details may be changed without departing from the invention, which is defined by the claims.

A method according to the present invention includes tDCS for sustained relief of indications such as tinnitus, epilepsy, addiction, depression, stroke, anorexia, pain, and/or the improvement of attention and/or motor learning. The discussion herein focuses primarily on the application for treating tinnitus, but the systems and methods may also be used for the treatment of other indications, including those listed above.

Treatment methods according to the present invention are non-invasive and can be delivered with a portable device that is quick and easy to use. The proposed methods of treating tinnitus do not require surgery, avoiding the risks and costs associated with an invasive procedure. As well, the proposed methods can be delivered without clinician intervention, such as by a patient at home, which is less expensive and time-consuming than traveling to treatment centers (e.g., rTMS). A system according to the present invention provides a novel portable tDCS device for fast, comfortable, and accurate electrode placement over any part of the cortex without the need to re-measure electrode location before each treatment session.

Treatment parameters may be combined to increase a duration and/or degree of changes in cortical excitability to produce sustained relief of tinnitus. Tinnitus patients usually exhibit cortical hyperactivity that can be corrected with non-invasive stimulation. Although tinnitus typically begins with a problem in the peripheral auditory system, these problems lead to complications in the central nervous system. In many cases, hearing loss can lead to changes in cortical plasticity, manifesting as cortical hyperactivity. This link between tinnitus and cortical hyperactivity has been confirmed by studies demonstrating reductions in tinnitus correlated with reductions in cortical hyperactivity. It has been discovered that non-invasive cortical stimulation may be used to treat tinnitus because stimulation can modulate cortical excitability to disrupt or reduce cortical hyperactivity. Methods according to the present invention may combine treatment parameters of tDCS to increase the duration and degree of changes in excitability, thus increasing the duration and/or degree of tinnitus relief.

Regular, such as daily, weekly, or monthly sessions of tDCS may be delivered to or over a therapeutic target region. The therapeutic target region is the area of the brain that is generally targeted for treatment of the various indications, although other areas may be tested and targeted for the desired treatments. For example, tDCS may be used to disrupt the cortical hyperactivity that occurs in tinnitus patients. As such, a primary target to receive stimulation may be the left temporoparietal area (LTA) because functional neuroimaging has revealed hyperactivity in this region in most (85-90%) patients regardless of the laterality of tinnitus (left-side, right-side, or bilateral). However, non-invasive stimulation to right temporoparietal area may be effective in some patients, and a recent study has suggested that in patients with unilateral tinnitus, non-invasive stimulation delivered to the temporoparietal area contralateral to the affected side may be more effective than ipsilateral or left-side stimulation.

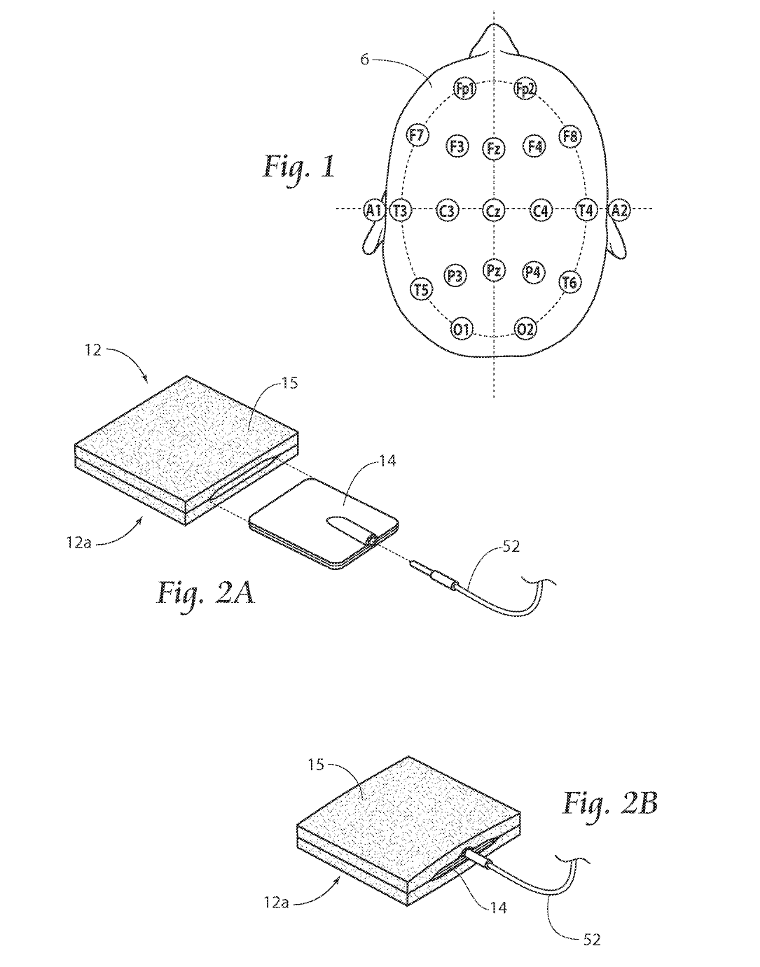

The locations of the therapeutic target regions can be determined using a landmark-based system. Specifically, a 10-20 EEG system may be used, which is an internationally recognized method that allows coordinates on the scalp based on anatomical landmarks to be correlated to cortical locations. In a 10-20 EEG system, the coordinates correspond to distances between adjacent electrodes being either about 10% or about 20% of the total front-back or right-left distance of the skull. The numbers in the coordinates identify the hemisphere location, and the letters identify the lobe (frontal, temporal, central, parietal, and occipital). The 10-20 EEG system coordinates are shown in FIG. 1. The location of the LTA can be approximated with the system coordinates C3 and T5, or P3 and T3.

Examples of possible therapeutic target regions of other indications are as follows: Left Dorsolateral prefrontal cortex (near coordinate F3) for the treatment of depression; Primary motor cortex (near C3 or C4) for pain; Dorsolateral prefrontal cortex (near F3 or F4) for addiction; Frontal cortical areas (near F3 and F4) for memory improvement; Primary motor cortex (near C3 or C4) of either the affected or unaffected hemisphere for motor rehabilitation and the language centers of the brain for treating aphasia of a patient who has suffered a stroke; the left dorsolateral prefrontal cortex (near F3) for treating attention issues; the primary motor cortex (near C3 or C4) for motor learning; anode left/cathode right over the prefrontal cortices (near F3 and F4) for treating anorexia; and for the treatment of epilepsy, the target area may be guided by the location of abnormal activity found on an EEG. The preferred therapeutic target regions for the various indications can be located using the 10-20 EEG system coordinates shown in FIG. 1.

Another therapy or treatment regime may consist of a 3-stage process: 1) a setup stage, where a therapeutic target region and a specific treatment location may be determined via a landmark-based system and confirmed through stimulation-evoked responses, 2) a priming stage of a predetermined number, e.g. 5, of regular sessions of tDCS to generate sustained relief of the symptom being treated, and 3) a maintenance stage, where periodic sessions of tDCS produce relief persisting during the interval between sessions. As stated above, the systems and methods of tDCS will be discussed with specific reference to the treatment of tinnitus, but the tDCS systems and methods disclosed may be used to treat a variety of other indications.

Subject, or patient, demographic data and medical histories may be obtained or utilized to determine eligibility for treatment and for post hoc analysis of factors for data categorization, etc. Eligible subjects may undergo a typical audiometric examination (hearing test, tinnitus pitch and intensity matching). At baseline, prior to stimulation, tinnitus intensity may be assessed using a scale such as a Numerical Rating Scale (NRS; 0=absence of tinnitus, 10=loudest sound imaginable) or a Visual Analog Scale (VAS), and tinnitus distress may be assessed using a generally accepted validated questionnaire, such as the Tinnitus Questionnaire. The Tinnitus Questionnaire is a generally accepted subjective questionnaire administered to determine the effect of tinnitus on their quality of life, including tinnitus effect on irritability, sleep, self esteem or perception, pain, and daily activities.

Methods according to the present invention allow one or more, but preferably a single, clinician to confirm correct electrode position quickly and accurately during the set-up phase. A method according to the present invention of determining correct or desired electrode position combines the simplicity and speed of a landmark-based system with stimulation-evoked responses to confirm the correct electrode position for tDCS to treat tinnitus. This method can be performed by one or more people, but preferably a single clinician without additional personnel, and will reduce the time and cost of determining correct electrode position compared to functional neuroimaging, which is presently required to obtain accurate electrode positioning for cortical stimulation.

In the setup stage, brief sessions of tDCS are used to produce responsive reductions in the problem symptom, such as tinnitus, or other neuro-response signals, such as changing from a baseline level of activity and/or the presence of a remote effect, indicating an effective electrode position. The response may be functional or non-functional. The setup stage may use sessions of anodal tDCS. Anodal tDCS increases cortical excitability and may be used to generate transient tinnitus relief. To determine correct or desired electrode position for tDCS, it is preferred to elicit transient reductions in tinnitus, and anodal tDCS is preferred over cathodal tDCS for this purpose, although cathodal tDCS may also produce transient reductions. tDCS affects cortical excitability in a polarity-dependent manner: anodal tDCS increases cortical excitability, while cathodal tDCS decreases excitability. Transient reductions in tinnitus may be produced via normalization of cortical hyper- or hypo-activity using non-invasive stimulation with parameters known to increase cortical excitability, including anodal tDCS. Thus, anodal tDCS is preferred to evoke transient reductions in tinnitus to determine correct or desired electrode location.

The brief sessions of tDCS may be limited to less than 20 minutes, and preferably to about 3 minutes to produce transient effects that will last less than or more than the stimulation time, or preferably ≦1 minute. When the brief sessions of anodal tDCS produces a responsive reduction in tinnitus by a predetermined amount (e.g. a certain percentage reduction or point reduction on a scale) as compared to pre-stimulation reports or previous reports based on the same scale, then correct electrode position will be logged and considered confirmed for the priming stage.

In preparation for the set-up phase of the treatment, the therapeutic target region, such as the left temporoparietal area (LTA), may be mapped on the subject's scalp using the 10-20 EEG system. A tape measure may be used to determine coordinates from the nasion (depression above bridge of nose) to the inion (lowest point on back of skull), as well as from the left to right preauricular points. Then, a skin marker may be used to mark the locations corresponding to the therapeutic target region, such as T3, C3, P3, T5, and the midpoint of C3 and T5 for locating the LTA.

The entire area defined by the EEG system coordinates associated with the therapeutic target region, as well as surrounding areas, may be cleaned and inspected for signs of irritation and lesions. Stimulation is preferably not delivered over any area exhibiting irritation or lesions to avoid causing damage to the scalp and discomfort to the patient. Furthermore, the area may be shaved, or the patient's hair cut, to provide better and more direct access to the scalp.

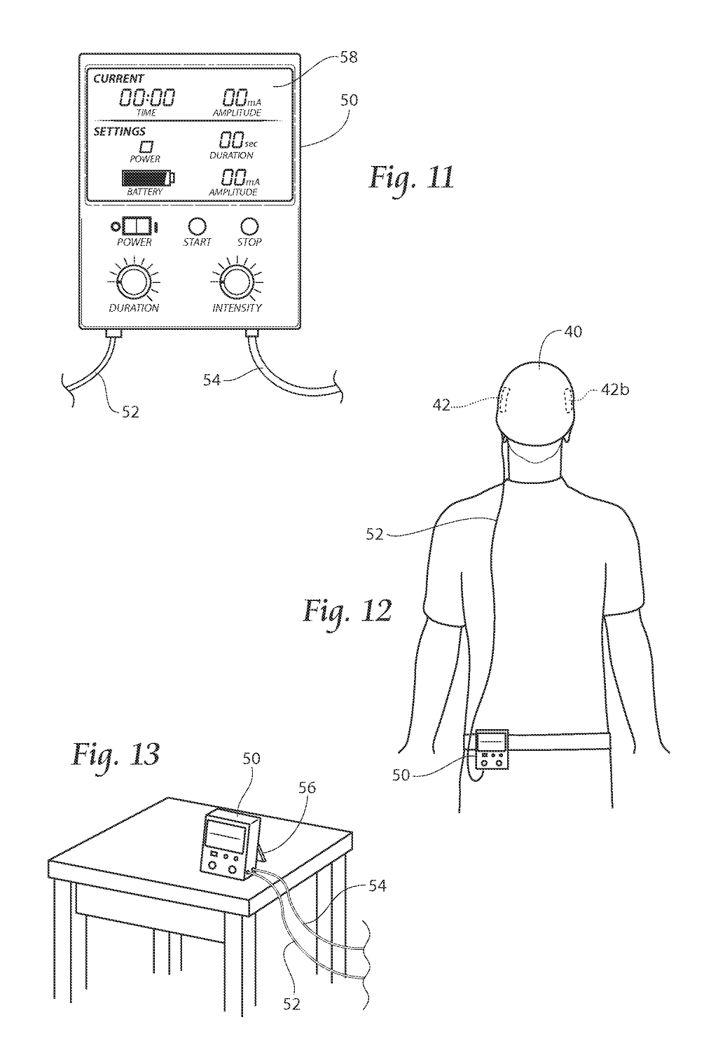

Stimulation may be provided via a primary electrode 12, such as the sponge electrode 12a shown in FIGS. 2A and 2B, wherein the stimulating portion 14 of the electrode is inserted or positioned behind a sponge portion 15 that will touch the patient's head. The sponge may be sized between 20 cm² and 35 cm². In a preferred embodiment, the sponge is 5 cm by 7 cm (35 cm²), though other sizes may be desired and used for certain applications of the invention. The sponge electrode may be secured to the subject's head by securing means, such as elastic straps, through treatment apparatuses such as the halo apparatus 20 shown in FIGS. 4-6, the electrode cap 30 shown in FIG. 9, the treatment cap shown 40 in FIG. 10, as discussed in further detail below, or other securing means. The sponge electrode 12a may be positioned and secured over the therapeutic target region, such as the LTA, and a second sponge electrode, such as a 35 cm² or 50 cm² sponge electrode, may be placed elsewhere on the patient, to serve as the return electrode 12r. In the embodiment shown in FIGS. 4-6, the second electrode 12r is placed and secured on the skin on the left shoulder. Alternatively, the return electrode 12r may be placed elsewhere on the scalp, at an operative distance from the primary electrode 12 to allow the electric field to diffuse.

Prior to stimulation, the sponge portions 15 on the sponge electrodes 12a may be soaked in ˜12 mL saline (0.9% NaCl) or a conductive gel. The sponge electrode 12a may further employ means for staying moist, such as an irrigation system that continuously, regularly, or intermittently delivers saline or conductive gel to the sponge to prevent the sponge from drying, and causing discomfort to the patient during stimulation.

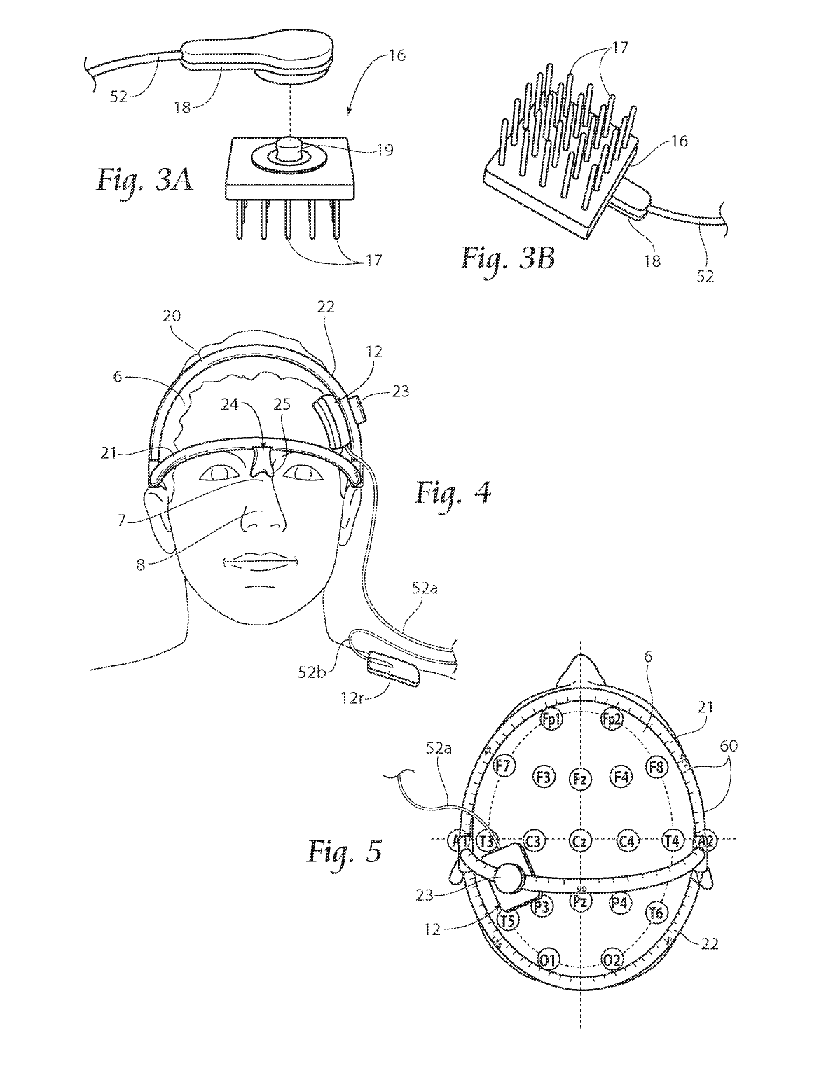

Alternatively, the present systems and methods may utilize dry electrodes, such as the dry electrode 16 shown in FIGS. 3A and 3B. A dry electrode 16, such as the one shown, may employ a plurality of surface micro-structures 17. These micro structures 17 preferably augment the electrode/skin interface by mechanically connecting the skin and the electrode 16, thus facilitating the transmission of the signals therebetween. The microstructures 17 may penetrate through one or more cutaneous layers of the scalp 6 to augment the electrode/skin interface. A benefit to the use of a dry electrode 16 is that the target area may not need to be shaved (as is preferred when sponge electrodes are used) prior to stimulation, as the micro structures 17 can contact the scalp by extending through the hair. Thus, the micro structures 17 are preferably long enough to reach through the hair to the scalp 6. The micro structures 17 may be small pins or micro-needles, or softer bristles having a conductive material. In the embodiment shown, the dry electrode has a plurality (e.g. 25) pins 17 of a desired length, such as about 0.25 inches. The pins 17 are preferably plated with Nickel and are electrically tied together, or in electrical communication with each other. The dry electrodes 16 may comprise separable elements wherein there are corresponding attachment means 18, 19 on a lead portion 52 and on the main electrode body that can be secured together for providing stimulation.

Alternatively, the present systems and methods may utilize microtubule electrodes (not shown) that make direct contact with the scalp 6 and deliver stimulation, similar to the dry electrode 16. However, a microtubule electrode embodiment may further deliver saline or other fluid through the microtubules, similar to structures 17 in the dry electrode 16. The saline or other fluid may be delivered from a fluid source and may be dispensed continuously throughout stimulation, or “on-demand” as necessary through a control mechanism that directs and allows fluid to pass from the source to the electrode microtubules.

The present systems and methods may use an impedance monitor, either as a tool separate from the electrical stimulation delivery apparatus, or constructed in conjunction with the delivery apparatus, to determine the impedance to current flow between the electrodes 12 and 12r. The impedance monitor may measure the voltage and current passing through the electrodes, and the impedance (Z) may be calculated by applying Ohm's Law (Z=V/I) to determine the electrode impedance. The electrode impedance may be important to the effectiveness of the methods according to the present invention, therefore the use of the impedance monitor can ensure that the electrodes are performing properly and determine whether a different electrode or electrode type may be necessary for the particular tDCS session.

During a set-up phase, threshold amplitudes may be determined for a patient, to determine a stimulation amplitude (Iₛₜᵢₘ) to be used. Threshold amplitudes for cutaneous perception (T_{perception}) are preferably measured by ramping up stimulation amplitude in desired steps, such as in increments of 0.1 mA, or continuously at any desired rate, such as at a rate of approximately 0.2 mA/second until the patient experiences tingling beneath the electrode, after which stimulation delivery is preferably discontinued. Additionally, a threshold amplitude for discomfort (T_{discomfort}) may be measured by ramping up stimulation amplitude in desired increments or at a desirable rate, such as about approximately 0.4 mA/second, until the patient experiences discomfort, after which the stimulation delivery is preferably discontinued. Stimulation amplitudes (Iₛₜᵢₘ) are then preferably set at a level below T_{discomfort}, such as between 75%-99% of T_{discomfort}. T_{discomfort} may also be determined or estimated based on the typical threshold values of individuals of the same attributes such as age, sex, race, etc., which may be correlated in a database. Such typical threshold of discomfort values may then be used to determine the stimulation amplitude (Iₛₜᵢₘ), which may be based on a comparison confidence level between patient attributes and database factors.

The figures show embodiments of an electrical stimulation system for providing tDCS according to the present invention. As shown, an electrode 12 may be placed over, preferably in electrical communication with the scalp 6 at a therapeutic target region, such as a left temporoparietal area (LTA), electrical stimulation through which will attempt to normalize the cortical hyper- or hypo-activity that occurs in this region for most tinnitus patients. As stated above, while the LTA is a typical therapeutic target region for the treatment of tinnitus, other successful treatments may be found by targeting other regions of the brain. If a desired change, such as a transient reduction in tinnitus, has not occurred, then the electrode may be moved in a desired pattern, such as 20 mm from the original location systematically in 4 directions (laterally, medially, anterior, posterior), and tDCS may be administered again at each location until the predetermined intensity reduction in tinnitus is achieved, reported or objectively or subjectively observed. In addition to these fine-tuning changes in location, more gross changes in electrode position may be made by moving to a different therapeutic target region. The various regions may be targeted during the set-up phase in any order determined by the clinician. Preferably, for example, the set-up phase for the treatment of tinnitus will first target the LTA, followed by the RTA (near the region defined by the coordinates C4-T4-T6-P4), then the left dorsolateral prefrontal cortex (near F3), followed by the right dorsolateral prefrontal cortex (near F4), to achieve the desired decrease in tinnitus. Another effective method may involve targeting the temporoparietal area and the dorsolateral area simultaneously to decrease tinnitus.

Therefore, the system according to the present invention is easily movable to allow for the clinician or individual performing the set-up stage to move the electrode position or switch easily to the right temporoparietal area, or other target area, if tDCS delivered to a tested region does not produce transient reduction in tinnitus. The ability to simply and quickly move the electrode, and thus target different therapeutic target regions for producing a transient response, or other neuro-response according to the indication being treated, will improve the likelihood of determining the correct electrode position.

FIGS. 4-6 show one embodiment of a suitable device according to the present invention. A halo apparatus 20 shown in FIGS. 4-6 allows for efficient electrode 12 positioning by having movable portions. While the halo apparatus 20 is particularly useful during the set-up stage, the same or similar halo 20 may also be used during the priming and/or maintenance stages. As shown, the halo 20 may have a main halo portion 21, a moveable arch 22, an electrode apparatus 23 to hold the primary electrode 12, a current source (shown in FIGS. 11-13) electrically coupled to the primary electrode 12 via a lead 52, and may be connected to a user interface used to set treatment parameters and control the current to the electrode 12. FIG. 4 shows return electrode 12r positioned on the patient's shoulder. In an alternative embodiment, a return electrode 12r may be positioned and/or supported on the halo apparatus 20, such as by a second electrode apparatus (not shown), similar to the electrode apparatus 23 shown, or may be a node or other extension formed onto the halo apparatus 20. The return electrode 12r positioned on the halo apparatus 20 may be stationary or may be moveable by slidable connection means on either the main halo portion 21 or the moveable arch 22.

As shown, the main halo portion 21 may be substantially ring-shaped, such as in the form of an ellipse, sized and configured to fit about at least a portion of a patient's head. Preferably, the halo portion 21 rests at a location slightly above eye-level. The halo portion 21 may further comprise positioning means 24, such as the nose piece 25 shown. The nose piece 25 preferably extends radially from the main halo portion 21 and is configured to rest on the bridge 7 of the patient's nose 8. The positioning means 24 may also be one or a pair of ear pieces that would rest on the patient's ear(s), or any other positioning means 24 that can serve to interface between the halo 20 and the patient's body, as well as provide a guide for accurate and consistent positioning on the patient's head each time the halo 20 is used. Preferred positioning means may provide for rotational, angular, and/or height registration.

Alternatively, the main halo portion 21 may be only a portion of a ring which surrounds only a portion of the patient's head, such as just the front of the patient's head, terminating at or near the patient's ears. This embodiment may require both a nose piece 25 and ear pieces to provide secure means for holding the halo apparatus 20 to the patient's head, or other suitable positioning means 24.

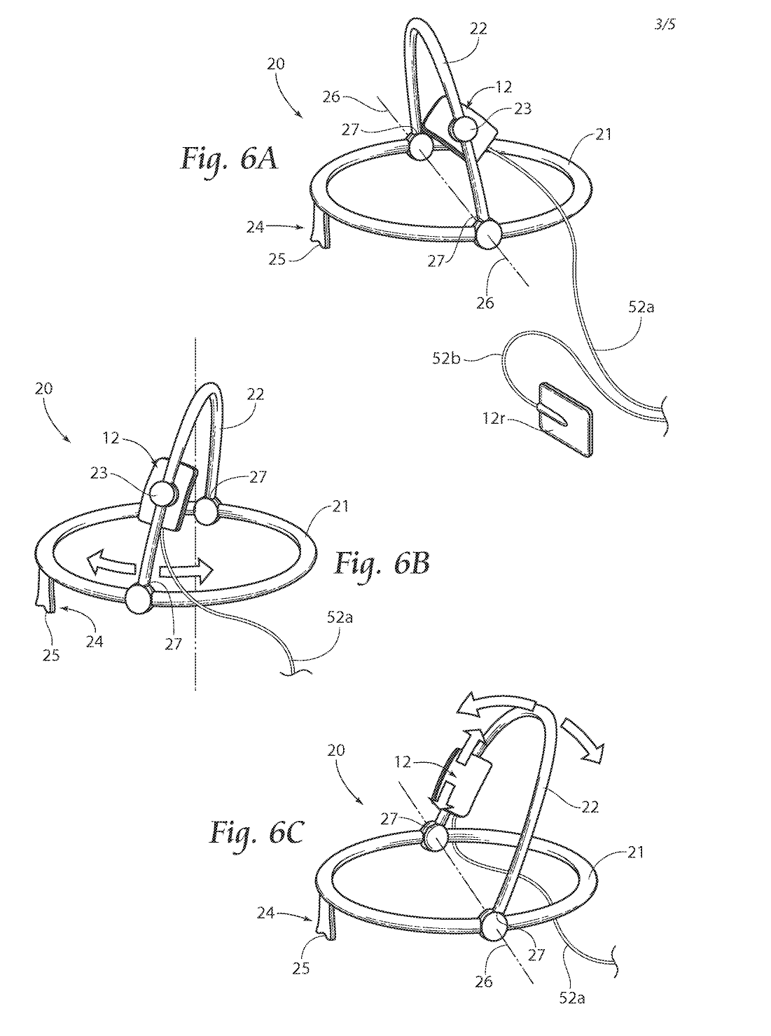

FIGS. 6A-6C depict the moveable nature of the halo apparatus 20. As shown in FIG. 6B, the moveable arch 22 may be slidably attached to the main halo portion 21 at its terminating ends 27, allowing spinning movement about the patient's head. As shown in FIG. 6C, the moveable arch 22 may also be rotatably attached to the main halo portion 21 at its terminating ends 27, allowing front-to-back or side-to-side movement about an arch tilting axis 26. Finally, the primary electrode 12, and/or a return electrode 12r, if provided on the halo apparatus 20, may be slidably attached, via the electrode apparatus 23, to move the electrode 12 and/or 12r along the moveable arch 22. Thus, the embodiment shown provides three-dimensional movement of the electrode 12 and/or 12r in order to provide efficient and simple movement of the electrode during the process of electrode positioning. However, portions of the halo apparatus 22 may be attached in a stationary fashion, eliminating at least one of the possible directions of movement of the electrode 12, such as eliminating the slidable nature of the arch 22 to halo 21 interface, and only providing capabilities for front to back or side to side movement (depending on the orientation at which the arch 22 is attached to the main halo portion 21), but not spinning movement. Alternatively, for example, another embodiment of the halo apparatus 20 may eliminate the rotatable attachment of the arch 22 to the main halo portion 21, and allow for only the spinning movement of the arch 22. Each of the moveable connection areas, i.e. the rotatable or sliding connection of the halo portion 21 to the moveable arch 22 may further comprise locking means 29 to retain the elements in their determined desired treatment position or during the testing of the various positions, or during priming or maintenance methods.

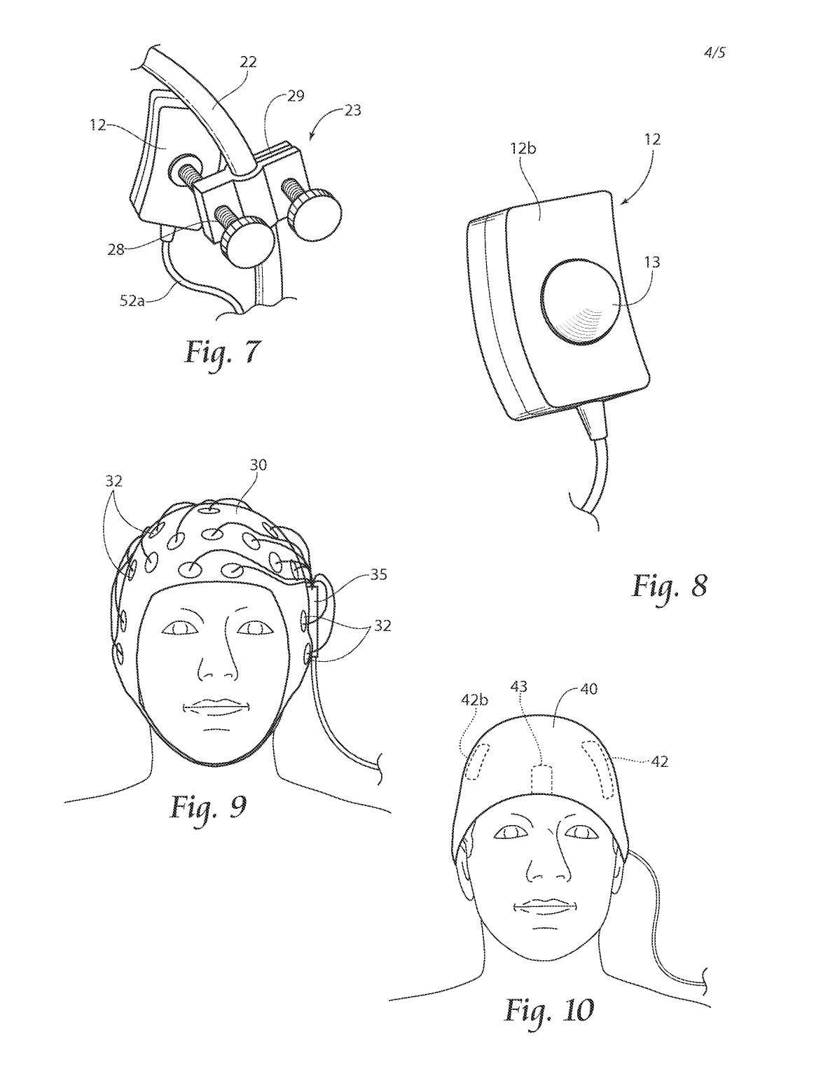

Furthermore, the electrode apparatus 23 may have means for positioning the electrode 12 (primary electrode 12 and/or return electrode 12r, if provided on the halo apparatus 20) nearer or farther from the moveable arch 22, in order to accommodate the touching relationship between the electrode 12 and the patient's scalp 6. As shown in FIG. 7, the electrode apparatus 23 may have a threaded member 28 which can be adjusted via rotation of the threaded member 28 to bring the electrode 12 into contact with the patient's scalp 6. FIG. 7 also shows a possible embodiment of the locking means 29 that may be used to secure the electrode apparatus 23 in a desired position on the moveable arch 22. Additionally or alternatively, the electrode 12 may be biased radially inwardly from the arch 22 by an electrode biasing member (not shown), such as a spring. In this manner, the electrode may be moved about a scalp while maintaining adequate contact therewith.

The present invention may also utilize an apparatus providing moveable continuous electrodes to quickly and easily move at least the primary electrode from one position to the next in order to generate a desired response. For example, the halo apparatus 20 may be automated to continuously move the electrode 12 in various positions about the patient's scalp 6. A rolling electrode 12b may be provided to accommodate such operability. To accommodate the mobility of the electrode 12b, the electrode 12b may comprise one or more rollerballs 13. The rollerballs 13 may allow the electrode 12b to easily roll or glide from one position to the next with limited friction or other resistance. Current may be delivered through the electrode rollerball 13 or the one or more rollerballs 13 may be attached to a sponge electrode through which the current is delivered, as shown in FIG. 8. An electrode comprising rollerballs 13 may be used alone, apart from the halo apparatus 20, or in connection with another moving electrode apparatus.

Additionally or alternatively, the moveable electrode concept may be implemented virtually via the grid electrode cap 30 shown in FIG. 9. As shown, the grid electrode cap 30 has a plurality of embedded electrodes 32 positioned about the cap 30 to cover a variety of positions on the patient's scalp 6. A control mechanism 35, such as a control panel, may be used to manually change the electrodes 32 to which the electrical signal is being transmitted, or the control mechanism 35 may run a program which continuously changes which of the embedded electrodes 32 is to serve as the primary electrode and/or which of the embedded electrodes 32 is to serve as the return electrode at a desired rate or in a desired pattern as selected and as input by the user. The grid electrode cap 30 may also have a stationary or shifting return electrode. Alternatively, a separate return electrode could be placed elsewhere on the patient's body, such as on the patient's left shoulder as shown in other embodiments.

The grid electrode cap 30, another embodiment of a grid electrode, or a plurality of independent electrodes may be used to implement a method of current steering during stimulation. During current steering, amplitudes of stimulation provided by the plurality of electrodes 32 may be independently varied, such as by control mechanism 35 or other manual operation of the current amplitudes. The superposition of electric fields generated by independently varied electrodes results in stimulation of an area between the two electrodes. The ratio of the amplitudes between nearby electrodes may be adjusted to “steer” the electric current to specific target areas, which may be useful for determining the most effective area for tDCS treatment.

While the grid electrode application is described as being a “cap”, similar head covering alternatives may be used, such as a helmet, a net covering (similar to a hair net), etc.

There may be a rest interval, such as 5 seconds to 10 minutes or more, between stimulation sessions to avoid carry-over effects. Once correct or desired electrode position has been confirmed, distances from the left preauricular point and the inion may be measured, and these distances may be noted, marked, and/or recorded to enable the electrode 12 to be placed in the same location across subsequent sessions. Additionally, once electrode placement is confirmed, a session of cathodal tDCS may follow, for approximately 5 to 30 minutes or more. The first session with a patient may take approximately 1-2 hours or less.

Upon completion of the set-up stage, the patient may begin the priming stage. In the priming stage, regular sessions, such as daily, weekly, or monthly, sessions of tDCS may be delivered to provide sustained tinnitus relief, which may last longer (in days) than the number of daily sessions implemented, e.g., ≧7 days, following the last session. The therapy may then progress to the maintenance stage where periodic sessions (e.g., 1-2 sessions every week or month) are used to produce lasting tinnitus relief, preferably for at least 3 months or up to 1 year or longer. Although the priming and maintenance stages may be applied without clinician intervention, such as at a patient's home, they may also be conducted completely within a clinician's office or by a clinician to minimize variables.

During the priming stage, sessions of tDCS repeated at daily, weekly, bi-weekly, monthly, or random intervals, may provide greater relief for longer durations than with single sessions. Delivery of these priming sessions, such as daily sessions, of tDCS may increase the duration and degree of tinnitus relief over present methods of tDCS to treat tinnitus. If tDCS is delivered in daily sessions, then the effects of tDCS accumulate and generate changes in excitability that are greater in duration and degree than the changes produced by an individual session. This, in turn, increases the duration and degree of tinnitus relief. Previous studies of tDCS to treat tinnitus have not delivered daily sessions in this manner.

As stated above, cathodal tDCS decreases cortical excitability and is preferred to generate sustained tinnitus relief. To produce sustained tinnitus relief, cathodal tDCS may be used during regular (e.g. daily, weekly, or monthly) treatment sessions.

tDCS may be delivered with a suitable device in a clinic or home setting according to the present invention. A system according to the present invention includes a novel portable tDCS system for fast, comfortable, and accurate electrode placement over any part of the cortex without the need to re-determine or re-measure electrode location before each session. This will allow patients to receive treatment without the inconvenience and cost associated with traveling to treatment centers. Such devices may be electrodes secured to the patient's head (e.g. by elastic straps) having the location of the electrodes secured to minimize or prohibit movement, the halo 20 shown in FIGS. 4-6, the electrode cap 30 shown in FIG. 9, or the treatment cap 40 shown in FIG. 10, or other suitable systems that guarantee proper electrode placement based upon the results of effective electrode position found during the set-up stage.

As stated above, the halo apparatus 20 used during the set-up stage may also be used during the treatment sessions during the priming stage. Once the proper electrode position is found through the brief sessions of, preferably anodal, tDCS treatment, the position of the electrode may be observed and recorded based on position markers 60, such as distance or degree markers on the halo main portion 21 and/or the moveable arch 22, shown on FIG. 5, such that the electrode 12 and/or 12r can be accurately positioned for subsequent treatment sessions. Alternatively, the electrode position may be marked directly onto the halo apparatus 21 or locked into position via the locking means 29 discussed above or another securing mechanism. The patient may then position the halo apparatus 21 on their head, using the positioning mechanism(s) 24 as a guide for proper placement, and begin treatment of the appropriate treatment area.

FIG. 10 shows an embodiment of a treatment device according to the present invention utilizing a custom made article of headwear having the electrodes accurately placed according to the correct electrode position determined during the set-up stage. The figure shows a treatment cap 40, which may be designed having a similar look and comparable comfort to a typical baseball cap or knit hat. As shown, a primary electrode 42 and a return electrode 42b are positioned and secured within the hat, and preferably, not visible from outside the cap 40. The cap 40 may have a battery-operated user interface to set the treatment parameters and a current source positioned within the cap 40, electrically connected to the electrodes 12 and/or 12r via lead 52. The cap 40 may include a position indicator 43, which may be on the outside of the cap 40, but preferably on the inside of the cap 40 as shown. The position indicator 43 may be configured to align with a feature of the patient's head, such as one or more of a nose, eye, eyebrow, ear, etc., to ensure accurate electrode placement. Alternatively, as shown in FIG. 12, the treatment cap 40 may have a single lead 52 extending from the treatment cap 40 to a unitary, battery-operated user interface and current source 50 that may be attached to a belt or other article of clothing. Therefore, the treatment may be provided by an entirely portable system that can be used in or outside of the home.

FIG. 13 shows a portable control panel and current source 50 having a stand 56 to position the panel 50 on a table or flat surface to be used during treatment in a stationary setting, if desired.

It is also contemplated that accurate electrode positioning for a priming stage may be effectuated through the placement of an implanted marker (not shown) under or in the patient's scalp 6. The implanted marker is preferably implanted during the set-up visit following the determination of the correct treatment electrode placement. The electrode 12 or apparatus used during the repeated priming sessions may have a marker sensing mechanism (e.g. magnetic or RFID) in order to obtain the accurate positioning of the electrode above the marker on the patient's scalp 6.

It is contemplated that the priming and maintenance stage treatments be implemented via a battery operated user interface and current source, such as the control panel 50 shown in FIG. 11. As shown in the figure, the control panel 50 has means for setting the parameters of the treatment such as duration and intensity of stimulation amplitude. A visual output, such as the screen 58 may indicate such values as the set parameters for the treatment as well the current values of the treatment parameters, or other values that may be provide useful operating information to the user, such as the electrode impedance measured by an impedance monitor. The control panel 50 may also implement treatment programs, such as programs involving a ramp-up, ramp-down period which slowly increases or the stimulation amplitude over a set period of time up to the set stimulation amplitude or down to 0 mA, in order to limit any discomfort to the patient.

The control panel 50 shown has a single lead 52 extending to the primary 12 and the return 12r electrodes, as well as a power source cord 54. In an alternative embodiment, the control panel 50 may be made more portable, and therefore, may be battery operated, eliminating the need for the power source cord 54.

Long-term reductions of tinnitus have been demonstrated according to the present invention using regular sessions, such as daily, weekly, or monthly sessions, of non-invasive stimulation having parameters that have demonstrably reduced cortical excitability. Relatively large amplitudes of tDCS may be used to achieve clinically significant tinnitus relief. To increase the duration and degree of tinnitus relief, tDCS may be employed at higher amplitudes than present methods of tDCS for tinnitus. The duration and degree of changes in cortical excitability induced by tDCS increase as amplitude increases, and behavioral effects studied with tDCS also increased with amplitude. Therefore, the present invention improves upon present methods of tDCS to treat tinnitus by increasing the amplitude from presently used levels, which are about one to about 1.5 milliamps (mA). Stimulation amplitude may be set at a value below T_{discomfort}, such as at about 75% to about 90% of the threshold for discomfort (T_{discomfort}), where T_{discomfort} generally may be between about 2 to about 3 mA. Such stimulation amplitudes are unlikely to cause adverse effects, as they are 2 orders of magnitude below limits generally accepted as causing brain lesions, and tDCS using current densities that are greater than the current density of 2-3 mA over a 35 cm² electrode, as an example of an electrode that may be used, generally have not caused adverse events.

Longer session durations of tDCS may be used to generate larger and longer-lasting relief of tinnitus compared to present methods of tDCS. The duration and degree of changes in cortical excitability and behavioral effects induced by tDCS increase as the duration of treatment session increases. By increasing the duration of sessions compared to the durations used in present methods of tDCS to treat tinnitus, which are typically 20 minutes, an increase in the duration and degree of tinnitus relief may be observed. tDCS has been delivered for 30-40 minutes, and side effects were limited to those normally observed with shorter sessions of tDCS. Thus, for tDCS, provided during the priming or maintenance stage, treatment sessions may last approximately 5 to 30 minutes or more.

The relatively weak constant current delivered during tDCS does not generate seizures or side effects associated with electroconvulsive therapy. Despite similarities in methodology, tDCS is not to be confused with electroconvulsive therapy (ECT). In ECT, high levels of current (≧800 mA) are delivered to the scalp to induce seizures, and patients are placed under anesthesia and given muscle relaxants to avoid pain and injury. On the other hand, tDCS delivers much lower levels of current (preferably ≦3 mA) to awake, as opposed to anesthetized, patients, and there are no known reports of seizures induced by tDCS in thousands of patients. Also, while ECT can cause severe adverse effects (e.g., memory loss, cognitive deficits), tDCS has had minimal adverse effects (e.g., skin irritation, fatigue, headache) in the treatment of tinnitus as well as other neurological disorders.

The sessions of tDCS during the priming stage may be repeated every day for a predetermined number of days (e.g. 2 to 5 consecutive days, or up to 30 days or longer), or the priming sessions may be repeated regularly or irregularly in a weekly, bi-weekly, or monthly intervals, or combinations thereof. During the interval between sessions, subjects may be asked to note any changes in tinnitus intensity and distress. Should a patient experience a significant reduction in tinnitus such that the symptoms are substantially or entirely relieved, or have reached a desired lower threshold, the patient may choose or may be directed to discontinue the priming stage sessions, and enter the maintenance stage, or end treatment all together. After the final session of tDCS, an assessment may be made as to the tinnitus distress currently being experienced by the patient, such as administration of the Tinnitus Questionaire, or having the patient provide a rating on the NRS or VAS as to the level of the symptoms. A follow up appointment may be held at a follow-up time, such as 1 week, after the final session, and effectiveness of the treatments and sustained relief may be assessed. Such assessment may be administered through the NRS, VAS, or the Tinnitus Questionnaire. Preferably, patients may be asked to keep track of changes in tinnitus intensity and distress at prescribed regular intervals or random intervals, such as every day for 2 weeks after treatment or after the follow-up time, or whenever the patient notices a change.

During the maintenance stages, therapy sessions may be provided at predetermined intervals, such as daily sessions where the session takes place at the same time everyday or weekly sessions performed on the same day(s) every week. Alternatively, the maintenance sessions may be performed as needed or at random times, perhaps under the direction to undergo, i.e., at least 1-2 sessions per week or a predetermined number of sessions per week or month as convenient.

If desirable, sham, or placebo, stimulation may be provided to a patient to further establish a conditional baseline, and to determine if any, some, or all of the tinnitus reduction was due to placebo effect. In a first sham series, the process of seeking the correct electrode location with actual tDCS may be followed by a number (e.g. 5) of daily sessions of tDCS using sham-stimulation. Then, after a time break (such as one month) from stimulation, to prevent carry-over effects, a subject may undergo a real-tDCS series, where actual tDCS is delivered to determine correct or desired electrode location and daily sessions of actual stimulation may be delivered.

For sham tDCS, stimulation may be ramped up to Iₛₜᵢₘ over 10 seconds and discontinued while the patient remains seated for the remainder of the stimulation time, e.g. 40 minutes, as cutaneous sensations are most often experienced during the first few seconds of tDCS. Immediately following tDCS (sham and actual), the NRS may be administered or utilized, and the site of stimulation may be reinspected for signs of irritation and lesions. During the sham series, preferably all test sites (e.g., original location+4 surrounding sites) will be tested with sham tDCS.

tDCS delivered for a number of consecutive days, or according to the prescribed or random daily, weekly, bi-weekly, or monthly schedule, may result in a clinically significant improvement. For example, successful treatment may be demonstrated by 1) reductions in scores on the Tinnitus Questionnaire being significantly greater with real tDCS than sham tDCS, and 2) scores on the Tinnitus Questionnaire with real tDCS being reduced by ≧10 points (out of 84) on the Tinnitus Questionnaire, which is considered to be a clinically meaningful change. Improvements in the duration and degree of tinnitus reduction may be due to verifying correct electrode position with transient reductions in tinnitus, delivering a plurality of priming sessions instead of a single session, increasing stimulation amplitude, and increasing session duration.

In addition, patients may rate tinnitus intensity every day for 2 weeks following the last treatment session and/or follow-up time to determine how tinnitus intensity varies over time and to determine how long tinnitus relief persists. These results may confirm a desirable interval, such as 1 week, between maintenance sessions.

The effectiveness of several tinnitus treatments may be negatively correlated with a patient's tinnitus duration. A statistical analysis may be performed to assess whether patients who have had tinnitus for less than a predetermined number of years have greater reduction in Tinnitus Questionnaire scores compared to baseline versus patients who have had tinnitus for greater than the predetermined number of years. This test may reveal if methods according to the present invention are more effective than other treatments for patients with long histories of tinnitus. The statistical analysis may also assess whether the present methods are more effective for patients falling within certain demographic groups, such as sex, race, age, etc, and could provide insight as to effective parameter settings for various demographic groups.

As well, several tinnitus treatments are less effective for patients with decibels of hearing loss (moderately-severe to profound hearing loss). A statistical analysis may be performed to determine if patients with ≧56 decibels of hearing loss have less of a reduction on Tinnitus Questionnaire scores than patients with <56 decibels of hearing loss. This test may reveal if methods according to the present invention are more effective than other treatments for patients with hearing loss.

The foregoing is considered as illustrative only of the principles of the invention. Furthermore, since numerous modifications and changes will readily occur to those skilled in the art, it is not desired to limit the invention to the exact construction and operation shown and described. While the preferred embodiment has been described, the details may be changed without departing from the invention.

We claim:

1. A method for determining preferred electrode positioning for the delivery of transcranial direct current stimulation (tDCS) for the treatment of neurological indications, the method comprising:
providing a stimulation system comprising:
a primary electrode;
a return electrode;
means for securing the primary electrode to a subject's scalp;
a current source in communication with the primary and return electrodes; and
a user input device in communication with the current source;
wherein said stimulation system is configured to allow movement of the primary electrode to various locations on the subject's scalp;
determining a therapeutic target region, chosen arbitrarily or based on prior knowledge of success;
positioning the primary electrode in a first position on the subject's scalp at or near the therapeutic target region;
providing electrical current from the current source to the primary electrode;
monitoring the subject for an indication of a response;
optionally re-positioning the primary electrode to an alternative position on the subject's scalp until the desired response is observed;
observing the indication of the desired response;
noting a placement position of the primary electrode at which the desired response was observed as a guide for the preferred electrode position for the treatment of the indication; and
determining the preferred electrode position located at or near the placement position.

2. The method of claim 1 wherein the neurological indication being treated comprises tinnitus.

3. The method of claim 2 wherein the response comprises a change in sound perception.

4. The method of claim 2 wherein the therapeutic target region comprises a left temporoparietal area.

5. The method of claim 1 wherein the re-positioning step comprises moving the primary electrode in increments between 10 mm and 30 mm from the first or the alternative position.

6. The method of claim 1 wherein the optional re-positioning step comprises moving the primary electrode to a second therapeutic target region correlated to a different cortical area of the brain.

7. The method of claim 4 wherein the optional re-positioning step further comprises moving the primary electrode to at least one of a right temporoparietal area, a left dorsolateral prefrontal cortex, and a right dorsolateral prefrontal cortex.

8. The method of claim 1 wherein the providing step comprises providing one or more brief sessions of tDCS to the first or an alternative position until the response is observed, wherein each session is less than about twenty minutes in length.

9. The method of claim 8 wherein the brief sessions of tDCS are 3-4 minutes in length.

10. The method of claim 8 wherein the response comprises a transient reduction in tinnitus.

11. The method of claim 8 wherein the response comprises a transient change in sound perception.

12. The method of claim 1 wherein the primary electrode comprises a sponge electrode soaked in saline.

13. The method of claim 1 wherein the primary electrode comprises a sponge electrode having conductive gel.

14. The method of claim 1 wherein the primary electrode comprises micro elements to electrically couple to the subject's scalp.

15. The method of claim 14 wherein the micro elements are microtubules configured to deliver conductive fluid therethrough.

16. The method of claim 1 wherein the means for securing the primary electrode to the subject's scalp comprises at least one strap that is configured to be removably positioned on the subject's head.

17. The method of claim 1 wherein the stimulation system comprises a halo apparatus comprising:
a halo main body configured in at least a partial ellipse, sized and configured to fit around at least a portion of a subject's head;
a moveable arch, coupled at its terminal ends to the halo main body, sized and configured to fit over a portion of the subject's head;
at least one electrode attachment mechanism coupled to the moveable arch or the halo main body;
a primary electrode supported by one of the at least one electrode attachment mechanisms; and
a halo positioning means configured to physically contact a feature of the subject's head.

18. The method of claim 1 wherein the stimulation system comprises a cap having a grid of electrodes supported by an inner surface of the cap, the cap being positionable on the subject's head to force the grid of electrodes into contact with the subject's scalp, the method further comprising the step of selecting the primary electrode from the electrode grid.

19. The method of claim 1 wherein the neurological indication being treated is selected from a group consisting of: epilepsy, addiction, depression, stroke, anorexia, pain, improvement of attention, and improvement of motor learning.

20. A method for treating a neurological indication with transcranial direct current stimulation comprising the steps of:
providing a stimulation system comprising:
a primary electrode;
a return electrode;
means for securing the primary electrode to a subject's scalp;
a current source in communication with the primary and return electrodes; and
a user input device in communication with the current source;
completing a set-up phase wherein a preferred electrode treatment position on a subject's scalp to be used during the treatment sessions is determined; and
completing a priming phase comprising a predetermined schedule of treatment sessions wherein the primary electrode is secured to the subject's scalp at the preferred electrode treatment position;
wherein said predetermined schedule of treatment sessions results in sustained improvement in the neurological indication being treated.

21. The method of claim 20 further comprising completing at least one maintenance session, wherein tDCS is provided at the preferred electrode treatment position to extend the sustained improvement in the neurological indication being treated and maintain the symptoms of the indication at or below a treatment threshold.

22. The method of claim 20 wherein the neurological indication to be treated comprises tinnitus.

23. The method of claim 18 wherein the sustained improvement lasts longer in duration than the duration of the priming stage.

24. The method of claim 22 wherein the predetermined schedule of treatment sessions during the priming stage comprises daily sessions of tDCS for at least five consecutive days.

25. The method of claim 22 wherein the predetermined schedule of treatment sessions during the priming stage comprises sessions of tDCS lasting at least 5 minutes in length.

26. The method of claim 21 wherein at least one of the at least one maintenance session occurs between 1-2 weeks of the priming treatment session or a previous maintenance session.

27. The method of claim 21 wherein the at least one maintenance session occurs after the indication being treated equals or exceeds the treatment threshold.

28. The method of claim 20 further comprising providing a second stimulation system to be used during the priming stage.

29. The method of claim 28 wherein the second stimulation system comprises a device having the primary electrode and return electrode substantially respectively positioned in the preferred electrode treatment position that was determined during the set-up phase.

30. The method of claim 29, wherein the device is created based on measurements taken of the subject.

31. The method of claim 28, wherein the second stimulation system is different than the stimulation system.

32. The method of claim 20 wherein in the set-up phase further comprises:
determining a therapeutic target region,
chosen arbitrarily or based on prior knowledge of success;
positioning the primary electrode in a first position on the subject's scalp at or near the therapeutic target region;
providing electrical current from the current source to the primary electrode;
monitoring a selected area of the subject's body for a response;
re-positioning the primary electrode to an alternative position on the subject's scalp until the desired response is observed;
observing the desired response; and
noting the position of the primary electrode at which the desired response was observed as a preferred electrode treatment position for the treatment of the indication.

33. The method of claim 20 wherein the neurological indication to be treated is selected from the group consisting of: epilepsy, addiction, depression, stroke, anorexia, pain, improvement of attention, and improvement of motor learning.

34. A stimulation system for the delivery of transcranial direct current stimulation for the treatment of a neurological indication, the system comprising:
a halo apparatus comprising:
a halo main body configured in at least a partial ellipse, sized and configured to fit around at least a portion of a subject's head;
a moveable arch, coupled at its terminal ends to the halo main body, sized and configured to fit over a portion of the subject's head;
at least one electrode attachment mechanism coupled to the moveable arch or halo main body;
a primary electrode supported by one of the at least one electrode attachment mechanism;
a halo positioning means;
a return electrode; and
a current source connected to the primary and the return electrode.

35. The stimulation system of claim 34 wherein the halo main body is configured in a complete circle and is sized and configured to fit around the subject's head.

36. The stimulation system of claim 34 wherein the moveable arch is rotatable about an arch rotation axis that extends between the terminal ends of the moveable arch.

37. The stimulation system of claim 34 wherein the halo main body extends around a halo axis, and wherein the moveable arch is spinnable about the halo axis.

38. The stimulation system of claim 34 wherein wherein the primary electrode is moveable about a hemispherical range of motion.

39. The stimulation system of claim 34 wherein the electrode attachment mechanism supports the primary electrode and the electrode attachment mechanism is slidably coupled to the moveable arch.

40. The stimulation system of claim 34 wherein the electrode attachment mechanism comprises a positioning means capable of moving the primary electrode towards and away from the subject's head to accommodate contact with the subject's scalp.

41. The stimulation system of claim 39 wherein the electrode attachment mechanism further comprises a securing means to secure the primary electrode in position along the moveable arch.

42. The stimulation system of claim 37 wherein the at least one terminal end of the moveable arch further comprises a securing means to secure the moveable arch in position along the halo main body.

43. The stimulation system of claim 36 wherein at least one terminal end of the moveable arch further comprises a securing means to secure the moveable arch in position about the arch rotation axis.

44. The stimulation system of claim 34 wherein the halo positioning means comprises a nose piece configured to rest on the bridge of the subject's nose.

45. The stimulation system of claim 34 wherein the halo apparatus further comprises a return electrode attached thereto.

46. The stimulation system of claim 34 wherein the primary electrode is a sponge electrode soaked in saline.

47. The stimulation system of claim 34 wherein the primary electrode is a sponge electrode having conductive gel.

48. The stimulation system of claim 34 wherein the primary electrode is a dry electrode comprising micro elements to electrically couple to the subject's scalp.
